(19) United States
(12) Patent Application Publication
(10) Pub. No.: US 2011/0086928 A1
(43) Pub. Date: Apr. 14, 2011

(54) MITIGATING OR ELIMINATING THE CARBON FOOTPRINT OF HUMAN ACTIVITIES

(76) Inventors: George A. Olah, Beverly Hills, CA (US); G.K. Surya Prakash, Hacienda Heights, CA (US)

(21) Appl. No.: 12/973,535

(22) Filed: Dec. 20, 2010

Related U.S. Application Data
(63) Continuation of application No. 12/466,120, filed on May 14, 2009.
Application No. 12/466,120 is a continuation-in-part of application No. 12/537,647, filed on Aug. 7, 2009.
Application No. 12/537,647 is a continuation of application No. 11/766,408, filed on Jun. 21, 2007, now Pat. No. 7,608,743.
Application No. 11/766,408 is a continuation-in-part of application No. 11/402,050, filed on Apr. 12, 2006, now Pat. No. 7,605,293.
(60) Provisional application No. 61/053,954, filed on May 16, 2008.
(60) Provisional application No. 60/671,651, filed on Apr. 15, 2005.
(60) Provisional application No. 60/763,678, filed on Jan. 30, 2006.

(51) Int. Cl. C07C 68/02; C07C 29/151; C07C 41/09; C07C 29/04; C07C 68/00; C07C 1/22; C07C 2/42; C07C 2/12; C07C 27/06

(52) U.S. Cl. 518/702; 568/884; 568/698; 568/895; 558/277; 585/324; 585/322; 585/329

(57) ABSTRACT

A method for neutralizing or reducing the carbon footprint from carbon dioxide emissions due to human activities related to the combustion or use of carbon containing fuels. This method includes an initial step of capturing carbon dioxide and then chemically recycling it to form and provide a permanent inexhaustible supply of carbon containing fuels or products, which subsequently can be combusted or used without increasing the carbon dioxide content of the atmosphere. Thus, the current lifestyles that rely extensively on conventional carbon containing fuels and products can continue indefinitely without harming the environment to preserve and even improve the earth's atmosphere for the benefit of future generations.

### CROSS REFERENCE TO RELATED APPLICATIONS

This application is a continuation of U.S. application Ser. No. 12/466,120 filed May 14, 2009, which claims the benefit of U.S. provisional application No. 61/053,954 filed May 16, 2008. the entire content of each of which is expressly incorporated herein by references thereto.

Additionally, this application is a continuation-in-part of U.S. application Ser. No. 12/537,647 filed Aug. 7, 2009, which is a continuation of application Ser. No. 11/766,408 filed Jun. 21, 2007, now U.S. Pat. No. 7,608,743, which is a continuation-in-part of U.S. application Ser. No. 11/402,050 filed Apr. 12, 2006, now U.S. Pat. No. 7,605,293, which claims the benefit of U.S. provisional application No. 60/671,651 filed Apr. 15, 2005 and U.S. provisional application No. 60/763,678 filed Jan. 30, 2006, the entire content of each of which is expressly incorporated herein by references thereto.

### BACKGROUND

In our 21^{st} century society, one of the major concerns frequently expressed is the environmentally harmful increase in the “carbon footprint” of human activities leave on our planet. The carbon footprint is the amount of carbon dioxide produced by an entity due to the use of carbon containing fuels or the like. The carbon dioxide that is inevitably formed from the combustion or degradation of carbon based fuels, materials or any live processes themselves is of major concern as it contributes significantly to the change of our climate by causing harmful global warming. Wide spread efforts are made suggesting to drastically reduce the use of carbon containing fuels and materials and to replace fossil fuels with non-carbon containing energy sources. It is emphasized that non-carbon containing sources of energy, notably alternative sources such as hydro, geothermal solar, wind as well as others and atomic energy should be increasingly used. In the foreseeable future, however, fossil energy sources will continue to play a major role due to its ready availability and relatively low cost compared to the alternatives.

The environmentally harmful cause of any carbon containing compound is that upon its combustion or degradation carbon dioxide is formed, which is a greenhouse gas. To put the problem into a context, the use of coal or other fossil fuels, power plants burning such fuels produce annually in excess of 30 billion tonnes of carbon dioxide, which is further augmented by large amounts due to varied industrial activities, petroleum and natural gas use, agriculture, live processes and discharge of varied natural sources. Even if the oceans and photosynthesis processes in nature can absorb and recycle as much as half of the CO₂ related to human activities, the remaining amount still greatly overloads and upsets our terrestrial CO₂ balance.

It is therefore emphasized, as reflected by the Kyoto agreement and other efforts, that we must drastically decrease our human activity generated CO₂. It is recognized that the use of non-carbon containing and renewable sources of energy, as well as capture and sequestration of carbon dioxide emissions at the bottom of the sea or underground should be used wherever feasible. There are, however, obvious limitations of these approaches. Sequestration besides being a costly process is only a temporary solution. Sequestered carbon dioxide may not stay long out of the atmosphere. Any geological disturbance (earthquake, slides, volcanic eruption, etc.) can cause instant release of huge amounts of deadly carbon dioxide, which being heavier than air could suffocate any living creature on a large scale.

There are other known ways to reduce the carbon footprint of an entity. The carbon footprint can be efficiently and effectively reduced by applying the following steps:
first accurately determining an entity's current carbon footprint;
identifying hot-spots in terms of energy consumption and associated CO₂-emissions where reductions may be possible;
optimizing energy efficiency to reduce of CO₂-emissions and reduction of other GHG emissions contributed from production processes; and
identifying solutions to neutralize the CO₂ emissions that cannot be eliminated by energy saving measures.
The last step includes carbon offsetting: the investment in projects that aim at the reducing CO₂ emissions, for instance biofuels or tree planting activities.

Whereas these all are sensible alternatives and approaches and the use of alternative energy sources and fuels can provide some relief, these are per se insufficient to provide a significant solution to the problem and new technological approaches are needed. Some new approaches are now provided by the present invention.

### SUMMARY OF THE INVENTION

The invention relates to a method for neutralizing or reducing the carbon footprint from carbon dioxide emissions due to human activities related to the combustion or use of conventional carbon containing fuels. This method includes an initial step of capturing carbon dioxide and then chemically recycling it to form and provide a new and permanently inexhaustible supply of carbon containing compounds in the form of fuels, synthetic hydrocarbons or other products, which fuels and products subsequently can be combusted or used without increasing the overall carbon dioxide content of the atmosphere. The invention also relates to the use of captured or recycled carbon dioxide to form such new carbon containing compounds or products to thus control and reduce the carbon dioxide emissions which result in the overall carbon footprint of the planet. Thus, the current lifestyles that rely extensively on conventional carbon containing fuels and products can continue indefinitely by substituting the new carbon containing compounds or products obtained by recycling of carbon dioxide without harming the environment to preserve and hopefully improve the earth's atmosphere for the benefit of future generations.

### DETAILED DESCRIPTION OF THE PREFERRED EMBODIMENTS

The following definitions apply to this invention.

A carbon footprint is a “measure of the impact human activities have on the environment in terms of the amount of green house gases produced, measured in units of carbon dioxide”. It is meant to be useful for individual, entities and organizations to conceptualize their personal (or organizational) impact in contributing to global warming. A conceptual tool in response to carbon footprints are carbon offsets, or the mitigation of carbon emissions through the development of alternative carbon sources projects such as solar or wind energy or carbon recycling by biological process such as reforestation. A carbon footprint can be seen as a subset of earlier uses of the concept of ecological footprints

An “entity” can be an individual, household or other group of people, a firm, company or other organization. In particular, the entity is one that generates or is capable of generating a carbon footprint due to the combustion of fossil fuel, industrial activities, and fine process of other generation of carbon dioxide.

Thus, the carbon footprint is a measure of the excess global amount of carbon dioxide (CO₂) and other carbon containing greenhouse gases emitted by an entity or accumulated over the full life cycle of a product or service.

Normally, a carbon footprint is expressed as a CO₂ equivalent (usually in kilograms or tonnes), which accounts for the same global warming effects of different greenhouse gases. Carbon footprints can be calculated from all carbon dioxide sources or can be restricted to the main source of carbon dioxide, which is that generated from the use of fossil fuels, industrial and varied like process. Carbon footprints can either consider only direct emissions (typically from energy used in the home, workplace, transport (including travel by cars, airplanes, rail and other public transport), or can also include indirect emissions (including CO₂ emissions as a result of goods and services consumed).

The present invention offers a feasible way to mitigate the carbon footprint caused by human activities by not limiting or prohibiting the use of carbon containing fuels for energy generation, production of transportation fuels and varied derived materials and products, but instead by preparing such fuels and related carbon containing products from carbon dioxide that is captured from plants that generate it or by the removal of carbon dioxide from the atmosphere. By capturing and chemically recycling CO₂ emissions, a neutral or in some case a negative carbon footprint is achieved. This is feasible by recycling preferentially higher concentrations of industrial and natural CO₂ sources and emissions but also by capturing and recycling an equivalent amount, or on occasion even greater amounts, of CO₂ directly from atmosphere or air itself.

Of course, nature itself recycles carbon dioxide through agricultural plants and trees, but the combustion and use of oil and other fossil fuels has simply overloaded the system so that it cannot keep up with the amounts of carbon dioxide that are generated. The invention recognizes this shortcoming and now seeks to assist nature in this admirable recycling project. By first capturing carbon dioxide from the environment, or at least by preventing further amounts from being discharged, and then by converting the captured carbon dioxide to a carbon based fuel or feedstock, future generations can continue to utilize such fuels and feedstocks as well as the products made from such chemicals, without causing further harm to the environment. Thus, future sources of these fuels and products can be provided without increasing the emission of carbon dioxide or its resulting carbon footprint. The products can be used in an environmentally neutral manner.

In particular, the CO₂ that is captured and recovered can be used to produce suitable and renewable fuels such as methanol or dimethyl ether as well their derived products and materials as disclosed in U.S. Pat. No. 5,928,806 and US Patent Applications US 2006/0235091, US 2006/0235088 and US 2007/054969, the entire content of each of which is expressly incorporated herein by reference.

In the methods disclosed in these patent documents, the carbon dioxide can be captured from flue or off-gases of coal or other fossil fuel burning plants, geothermal power facilities, cement, aluminum or other industrial plants or factories, industrial or agricultural wastes or byproducts of natural gas production. The carbon dioxide also can be captured and removed from the air or atmosphere by absorbing it onto a suitable adsorbent followed by heating or otherwise treating the adsorbent to release the adsorbed carbon dioxide therefrom.

Preferably, the captured carbon dioxide is chemically recycled into methanol by various hydrogenative reductive processes. Other products, such as dimethyl carbonate, can be formed by reaction of the methanol with phosgene or by oxidative carbonylation of methanol. Advantageously, the methanol can be dehydrated to convert it to dimethyl ether which then can be used as is or as a starting material to form additional products. Dimethyl ether can be used as a substitute for natural gas and LPG for heating purposes for households or industrial use. Dimethyl ether can be heated in the presence of an acidic-basic or zeolitic catalysts to form ethylene or propylene. The ethylene or propylene can then be converted either to higher olefins, synthetic hydrocarbons or aromatics and their products, for use as feedstocks for chemicals or as transportation fuels. The ethylene or propylene can be hydrated to form ethanol or propanol and specifically isopropranol. These carbon based fuels and products then can be conventionally combusted or utilized without increasing the carbon footprint of any individuals or entities and without causing any further harm to the atmosphere by emitting any further carbon dioxide. All these processes are generally known from the prior patent documents mentioned herein, but the generation of fuels and products only after collecting or capturing carbon dioxide is a novel concept that will prevent further environmental damage while not changing the lifestyles to which the population has become accustomed. It also does not rely upon conservation or a reduction of the use of carbon based materials.

The present invention thus achieves mitigation of the harmful generation of CO₂ that adds to the carbon footprint of human activities through the initial capture of the equivalent or excess amount of carbon dioxide that is currently generated and by chemically recycling it preferably by using a suitable form of conversion to methanol or dimethyl ether making the human carbon footprint neutral or even in cases negative. These materials can then be used as convenient energy storage and transportation materials, fuels (including for internal combustion or fuel cells), household and industrial gases (for heating, cooking, etc.) as well as renewable raw materials for producing synthetic hydrocarbons and their products. By mitigating the harmful excessive carbon footprint of human activities, no excessive carbon dioxide is released into the atmosphere allowing the continued environmentally friendly renewable use of carbon containing fuels and materials while also diminishing or neutralizing the harmful environmental effect of excessive CO₂ discharge into the atmosphere causing global warming. At the same time it is assuring humankind of an inexhaustible, renewable and environmentally benign carbon source, namely, carbon dioxide, that is the starting point for making the fuels and products that are relied upon so heavily by everyone.

What is claimed is:

1. A method for neutralizing or reducing carbon dioxide emissions due to human activities related to the combustion or use of carbon containing fuels, which comprises capturing carbon dioxide and chemically recycling it to form carbon containing compounds or products which can be combusted or used without increasing the carbon dioxide content of the atmosphere.

2. The method of claim 1, wherein the carbon dioxide is captured from flue or off-gases of coal or other fossil fuel burning plants, geothermal power facilities, cement, aluminum or other industrial plants or factories, industrial or agricultural wastes or byproducts of natural gas production.

3. The method of claim 1 wherein the carbon dioxide is captured and removed from the air or atmosphere.

4. The method of claim 3, wherein the available carbon dioxide source is the atmosphere and the carbon dioxide is obtained by absorbing atmospheric carbon dioxide onto a suitable adsorbent followed by treating or heating the adsorbent to release the adsorbed carbon dioxide therefrom.

5. The method of claim 1, wherein the carbon containing compounds are carbon containing fuels or synthetic hydrocarbons.

6. The method of claim 1, wherein the captured carbon dioxide is chemically converted into methanol by hydrogenative reductive processes.

7. The method of claim 6, which further comprises dehydrating the methanol that is produced under conditions sufficient to produce dimethyl ether.

8. The method of claim 6, which further comprises converting the methanol or dimethyl ether in the presence of an acidic-basic or zeolitic catalysts to form ethylene or propylene.

9. The method of claim 8, which further comprises converting the ethylene or propylene either to higher olefins, synthetic hydrocarbons or aromatics, or their products for use as feedstocks for chemicals or as transportation fuels.

10. The method of claim 8, which further comprises hydrating the ethylene or propylene to form ethanol, propanol or isopropanol.

11. The method of claim 7, wherein the dimethyl ether is used as a substitute for diesel fuel, natural gas or LPG for heating purposes for households or industrial use.

12. The method of claim 6, which further comprises forming dimethyl carbonate by reaction of the methanol with phosgene or by oxidative carbonylation of the methanol
